(19) United States
(12) Patent Application Publication
(10) Pub. No.: US 2014/0294925 A1
(43) Pub. Date: Oct. 2, 2014

(54) FORMULATIONS COMBINING RAMOPLANIN AND RHAMNOLIPIDS FOR COMBATING BACTERIAL INFECTION

(71) Applicant: AGAE Technologies LLC, Corvallis, OR (US)

(72) Inventor: Xihou Yin, Corvallis, OR (US)

(73) Assignee: AGAE Technologies LLC, Corvallis, OR (US)

(21) Appl. No.: 14/201,633

(22) Filed: Mar. 7, 2014

Related U.S. Application Data
(60) Provisional application No. 61/775,336, filed on Mar. 8, 2013.

(51) Int. Cl. A61K 31/7028; A61Q 11/00; A61K 8/60; A61Q 15/00; A61K 8/14; A61K 8/64; A61K 38/15; A61Q 1/06

(52) U.S. Cl. CPC A61K 31/7028; A61K 38/15; A61Q 11/00; A61Q 1/06; A61Q 15/00; A61K 8/14; A61K 8/64; A61K 8/60
USPC 424/450; 514/2.9; 424/49; 424/64; 424/65; 536/18.2

(57) ABSTRACT

Provided herein are methods of treating a superbug infection in a subject that include administering to the subject a therapeutically effective dose of ramoplanin in combination with a therapeutically effective dose of one or more rhamnolipids, thereby treating the superbug infection. The superbug may be vancomycin-resistant Enterococcus, Clostridium difficile, or multidrug-resistant Clostridium difficile. The one or more rhamnolipids may include monorhamnolipid Rha-C₁₀-C₁₀, dirhamnolipid Rha-Rha-C₁₀-C₁₀, or monorhamnolipid Rha-C₁₀-C₁₀ and dirhamnolipid Rha-Rha-C₁₀-C₁₀. Also disclosed are pharmaceutical compositions that include ramoplanin, one or more rhamnolipids, and a pharmaceutically acceptable carrier, as well as formulations for preventing superbug infection.

### CROSS REFERENCE TO RELATED APPLICATIONS

The present application claims priority to U.S. Patent Application No. 61/775,336, filed Mar. 8, 2013, entitled “FORMULATIONS COMBINING RAMOPLANIN AND RHAMNOLIPIDS COMBATING BACTERIAL INFECTION,” the disclosure of which is hereby incorporated by reference in its entirety.

### TECHNICAL FIELD

Embodiments herein relate to pharmaceutical and biomedical uses of rhamnolipids, and in particular, to methods of using rhamnolipids for the synergistic enhancement of the antibiotic properties of various compounds when used to treat superbug infections.

### BACKGROUND

Vancomycin-resistant Enterococci (VRE), which are difficult to treat, are considered some of the most important nosocomial pathogens that cause infections in US hospitals. Enterococci, including VRE, are facultatively anaerobic, catalase-negative, Gram-positive Enterococci that are able to survive extreme temperatures and harsh chemical disinfectants, making them difficult to eradicate from environmental surfaces in hospitals and clinics. Effective treatments for bloodstream and deep-seated VRE infections have proved elusive.

Clostridium difficile (C. difficile) is a Gram-positive, anaerobic, spore-forming, and toxin-producing bacterium that produces heat-resistant spores that are exceedingly difficult to kill. C. difficile infection (CU) is characterized by intense intestinal, systemic, inflammatory reactions exhibiting symptoms ranging from mild diarrhea to severe colitis. C. difficile is the leading cause of nosocomial infections and diarrhea in hospitalized patients and long-term care facilities for the elderly. C. difficile now rivals MRSA as the most common organism to cause health care-associated infections in the United States, and it is estimated that 500,000 cases of C. difficile-associated diarrhea (CDAD) occur each year in the US. Metronidazole and vancomycin are the first-line clinical antibiotics to treat CDAD, although neither of them is fully effective.

Methicillin-resistant Staphylococcus aureus (MRSA) is refractory to many common clinic antibiotics such as methicillin, oxacillin, penicillin and amoxicillin. Vancomycin, formerly a golden standard treatment for MRSA, has become increasingly ineffective because of the emergence of vancomycin-resistant and vancomycin-intermediate Staphylococcus aureus (VRSA and VISA). Two novel antibiotics with unique mechanisms of action have been approved for clinical use to treat MRSA and/or VRE, but bacterial strains resistant to these agents have been encountered in the clinic already.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates the chemical structure of ramoplanin, in accordance with various embodiments;

FIG. 2 illustrates the chemical structure of representative rhamnolipids, in accordance with various embodiments;

FIG. 3 illustrates the HPLC profile of 95% pure rhamnolipids R95M, in accordance with various embodiments;

FIG. 4 illustrates the HPLC profile of 95% pure rhamnolipids R95D, in accordance with various embodiments; and

FIG. 5 illustrates the HPLC profile of 95% pure rhamnolipids R95DM, in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical contact. However, “coupled” may also mean that two or more elements are not in direct contact with each other, but yet still cooperate or interact with each other.

For the purposes of the description, a phrase in the form “NB” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Unless otherwise explained, the terms “rhamnolipids,” “rhamnolipid congeners”, “rhamnolipid components,” “rhamnolipid constituents,” and “rhamnolipid molecules” all have the same meanings as commonly understood by one of ordinary skill in the art. It is to be understood further that similar or equivalent rhamnolipid preparations described herein may be used in the practice of this disclosure, regardless of rhamnolipid origin, purity and composition. Similarly, three components of naturally occurring lipoglycopeptide antibiotic ramoplanins are regarded as mutually exchangeable and replaceable in the practice of this disclosure by one of ordinary skill in the art.

### ABBREVIATIONS

API: active pharmaceutical ingredient;

ATCC: American type culture collection;

BCS: Biopharmaceutics classification system;

CA-MRSA: community acquired methicillin-resistant Staphylococcus aureus;

CD: Clostridium difficile;

CLSI: clinical and laboratory standards institute;

Dap: daptomycin;

FIC: fractional inhibitory concentration;

FICI: fractional inhibitory concentration index;

GISA: glycopeptide intermediate Staphylococcus aureus;

HLB: hydrophilic lipophilic balance;

HPLC: high performance liquid chromatography;

LC-MS: liquid chromatography-mass spectrometry;

LPS: lipopolysaccharide;

MDR: multidrug-resistant;

MIC: minimum inhibitory concentration;

MRSA: methicillin-resistant Staphylococcus aureus;

MS/MS: tandem mass spectrometry;

PAH: polycyclic aromatic hydrocarbon;

R95D: 95% pure, dirhamnolipid congener Rha-Rha-C₁₀-C₁₀ predominant rhamnolipid sample;

R95DM: 95% pure, dirhamnolipid congener Rha-Rha-C₁₀-C₁₀ and monorhamnolipid congener Rha-C₁₀-C₁₀ predominant rhamnolipid sample;

R95M: 95% pure, monorhamnolipid congener Rha-C₁₀-C₁₀ predominant rhamnolipid sample;

TLC: thin layer chromatography;

Tei: teicoplanin;

Van: vancomycin;

VISA: vancomycin intermediate Staphylococcus aureus;

VRE: vancomycin-resistant Enterococci;

VRSA: vancomycin-resistant Staphylococcus aureus.

Embodiments herein provide methods of using rhamnolipid biosurfactant molecules in conjunction with various antibiotic compounds to combat drug-resistant, Gram-positive bacterial pathogenic infections or so-called “superbugs,” particularly global, life-threatening superbug infections. Various embodiments herein are based on the discovery of certain extraordinary synergistic effects of the antibiotic ramoplanin when in used in combination with rhamnolipids. In various embodiments, these synergistic effects may enable an effective treatment for superbug infections, such as vancomycin-resistant Enterococcus (VRE) and multidrug-resistant Clostridium difficile (MDR-CD).

Rhamnolipids are small molecule natural products of microbial origin whose antibacterial and antifungal activities are well documented. Due to the nature of rhamnolipids' antibiotic activities, their potential to be utilized as active pharmaceutical ingredients (API) and excipients had been ignored and/or avoided prior to the present disclosure. In pharmaceutical formulation practice, excipients are normally chosen due to their low activity or lack of activity. As disclosed herein, rhamnolipids are an exception to this conventional wisdom.

Rhamnolipids possess outstanding surfactant properties along with notable antimicrobial activities at high concentrations. As disclosed herein, when used as a biosurfactant, amphipathic rhamnolipids may dramatically increase the aqueous solubility, biomobility and bioavailability of poorly soluble substances, such as hydrophobic antibiotics and polycyclic aromatic hydrocarbons (PAHs).

In various embodiments, as described herein, the anti-superbug activities of highly water-soluble lipoglycopeptide antibiotic ramoplanin are dramatically increased when combined with rhamnolipids R95M. As further disclosed herein, the minimum inhibitory concentrations (MICs) for rhamnolipids R95M, R95D and R95DM were determined with exemplary superbugs, and ranged from 125 to 500 μg/ml. The MICs for ramoplanin alone were also determined with the same superbug strains, and were found to vary from 0.25 to 2 μg/ml. Thus, as described herein, synergistic activities result from the use of ramoplanin in combination with rhamnolipids against various superbugs, such as VRE and MDR-CD. For example, the MIC for ramoplanin against the multidrug-resistant C. difficile ATCC-BAA1382 was decreased to as low as 0.0125 μg/ml in the presence of 20 μg/ml of rhamnolipids R95M.

Additionally, as disclosed herein, the inhibitory efficacies of rhamnolipids, themselves, against various superbugs were shown to depend on their purity, composition and concentrations. Specifically, in various embodiments, purity, correct dosage, and composition were all shown to be important in achieving desirable antibiotic effects, either in application alone or in combination with other antibiotics.

As used herein, the term “superbug” refers to a drug-resistant, Gram-positive bacterial pathogenic infection, such as infection with vancomycin-resistant Enterococci (VRE), which are difficult to treat, and are considered some of the most important nosocomial pathogens causing infections in US hospitals. Enterococci, including VRE, are facultatively anaerobic, catalase-negative, Gram-positive bacteria that are able to survive extreme temperatures and harsh chemical disinfectants, making them difficult to eradicate from environmental surfaces in hospitals and clinics. Since the first VRE strains were isolated in France in 1986, VRE emerged in two distinct waves in the United States. The first wave was characterized by the prevalence of E. faecalis, which accounted for 90-95% of clinical enterococcal isolates, whereas the second wave consisted primarily of E. faecium. The ratio of clinically isolated E. faecalis vs. E. faecium has evolved from 9:1 in the 1970s to the current ratio of 1.8:1. The rate of vancomycin resistance among E. faecium escalated from 57.1% in 2000 to 80.7% of all bloodstream E. faecium infections in 2010. E. faecium has been rated as an emergent and challenging nosocomial problem.

Resistance determinants in VRE are typically classified into intrinsic and acquired resistances. Intrinsic resistance is defined as a non-transferable property, and is due to either a lack of target sites for the antimicrobial drug or insufficient ability of the drug to reach the target cell site. Acquired resistance results from either chromosomal mutations or genetic exchange of mobile elements like transposons or plasmids. Acquired resistance is common in E. faecium and E. faecalis, conferring resistance to multiple drugs such as vancomycin, erythromycin, gentamicin, kanamycin, streptomycin, and tetracycline. Different gene clusters (vanA and vanB are the most clinically relevant) are involved in the mechanism of vancomycin resistance.

Treatment options for VRE are extremely limited. Only linezolid and nupristin/dalfopristin (Q/D) are FDA-approved for VRE infection, although these two drugs have important limitations for treatment of severe VRE infection. Q/D was the first compound approved by the FDA for the treatment of VRE (E. faecium). However, Q/D is now infrequently used in clinical practice due to its metabolic interactions, adverse-effect profile, need for central vein administration, and issues with efficacy. Moreover, Q/D is not active against E. faecalis.

Linezolid is the second compound approved by FDA for the treatment of VRE infections. Linezolid is available for intravenous and oral administration and possesses excellent bioavailability. Linezolid is bacteriostatic, and its use in severe infections caused by VRE is limited by the frequent occurrence of clinical failures and recurrences. In addition, the toxicity profile of linezolid proscribes its use for infections that require prolonged therapy.

Two newer drugs with in vitro anti-VRE activity are daptomycin and tigecycline, but they are not FDA approved for VRE infection, and they also have important shortcomings in clinical practice. For example, daptomycin has become a front-line antibiotic for treating VRE infections, due to its in vitro bactericidal activity, although the clinical efficacy of this compound is hampered due to development of in vivo resistance. Thus, the optimal treatment for systemic and/or deep-seated VRE infections remains unknown.

Another pathogen encompassed by the term “superbug” is Clostridium difficile (C. difficile), a Gram-positive, anaerobic, spore-forming, and toxin-producing bacterium that produces heat-resistant spores that are exceedingly difficult to kill. C. difficile infection (CDI) is characterized by intense intestinal, systemic, inflammatory reactions exhibiting symptoms ranging from mild diarrhea to severe colitis. C. difficile is the leading cause of nosocomial infections and diarrhea in hospitalized patients and long-term care facilities for the elderly. C. difficile now rivals MRSA as the most common organism to cause health care-associated infections in the United States, and it is estimated that 500,000 cases of C. difficile-associated diarrhea (CDAD) occur each year in the US. C. difficile causes diarrhea linked to 15,000-20,000 American deaths each year. In recent years, the hypervirulent North American pulsed-field type 1 (NAP1/BI/027) strain of C. difficile is believed to have caused most of the reported increases in disease incidence and severity. A conservative estimate of the annual U.S. costs for CDAD management is $3.2 billion.

Metronidazole and vancomycin are the first-line clinical antibiotics to treat CDAD, although neither of them is fully effective. Fidaxomicin is the first antibiotic approved for the treatment of CDAD since oral vancomycin in 1986. Vancomycin is poorly absorbed when administrated orally. The use of metronidazole is associated with increased rates of treatment failure and relapse. In an acute CDI mouse model, fidaxomycin did not prevent recurrence of CDI. The challenge in dealing with CDI stems from the declined response to front line antibiotics, frequent treatment failure, and high rate of recurrence or multiple recurrences of infection following antibiotic therapy. The economic costs associated with recurrent CDI are estimated to exceed $13,000 per relapse.

Another “superbug” is methicillin-resistant Staphylococcus aureus (MRSA), a type of S. aureus that is resistant to certain antibiotics. MRSA is refractory to many common clinic antibiotics such as methicillin, oxacillin, penicillin and amoxicillin. Vancomycin, formerly a golden standard treatment for MRSA, has become increasingly ineffective because of the emergence of vancomycin-resistant and vancomycin-intermediate Staphylococcus aureus (VRSA and VISA). The Staphylococcus aureus strain ATCC700787 was characterized as both a MRSA and VISA strain.

Vancomycin-intermediate/resistant Staphylococcus aureus (VISA/VRSA) are specific types of antimicrobial-resistant bacteria that have become resistant to the glycopeptide antibiotic vancomycin. VISA and VRSA differ in their resistance levels to vancomycin. VISA's minimum inhibitory concentration (MIC) for vancomycin varies from 4 to 8 μg/ml, while VRSA's MIC for vancomycin is ≧16 μg/ml. From 2002 to 2012, thirteen VRSA cases have been confirmed in the US. Vancomycin is usually reserved for treatment of serious infections, including those caused by multidrug-resistant Staphylococcus aureus.

The steady increase of antimicrobial resistance is an evolutionary response to wide-spread antibiotic use. Antibiotic-resistant Gram-positive bacterial pathogens, including vancomycin-resistant enterococci (VRE) and Clostridium difficile infection (CU), have become serious concerns for global public health. Vancomycin was introduced to clinical use in the mid-1950s and became the choice of last resort to treat life-threatening and drug-resistant Gram-positive infections. Since the first report of vancomycin resistance in the clinic in 1988, vancomycin-resistant Enterococci (VRE) infections have become increasingly common. Since 2000, two novel antibiotics with unique mechanisms of action have been approved for clinical use to treat MRSA and/or VRE: linezolid, a synthetic oxazolidinone, and daptomycin, a lipopeptide natural product. Although resistance remains rare, bacterial strains resistant to these agents have been encountered in the clinic.

As described herein, the combination of ramoplanin and rhamnolipids produces a synergistic effect. The outcome of in vitro activity of a combination of two compounds may be classified into three distinct groups: additive effect (no interaction between compounds), synergistic effect (beneficial or favorable interaction between compounds), and antagonistic effect (adverse interaction between compounds). Antagonistic effects produce an inferior result compared to each compound when used as monotherapy. In contrast, synergistic effects produce a superior result when compared to each compound used independently.

Checkerboard, fractional inhibitory concentration (FIC), and time-kill analysis methods are commonly used to determine the effects of two compounds in an antimicrobial assay. The synergistic, additive, and antagonistic effects of one antibiotic in combination with a second antibiotic or a non-antibiotic drug/excipient may be defined by the values of the fractional inhibitory concentration index (FICI), which are 0.5, between 0.5 and 2, and >2, respectively.

The antibiotic combination treatments described herein may have great benefits over monotherapy with respect to efficacies, dosages, toxicity, and/or costs. In various embodiments, the combinatorial treatments may prevent the occurrence of resistance, kill multiple microbial infections, generate synergistic activity, decrease minimum inhibitory concentration and toxicity, and/or provide alternative therapeutic options.

As described herein, ramoplanin, a cell-wall active lipoglycodepsipeptide antibiotic, stands out from other antibiotics, including glycopeptide antibiotics vancomycin, teicoplanin and lipopeptide antibiotic daptomycin, by showing remarkable decreases in its minimum inhibitory concentration in vitro when used in combination with naturally produced rhamnolipid biosurfactants. Ramoplanin is poorly tolerated after intravenous or intramuscular administration. Also, ramoplanin is unstable in the bloodstream and not absorbed systemically when it is orally administered. However, the synergistic activity of ramoplanin in combination with rhamnolipids not only improves the efficacy of ramoplanin, but may also help overcome its shortcomings and enable its use as a frontline clinic drug against superbug infections.

In order to make the various embodiments disclosed herein more easily understood, explanations are provided below for several terms.

Surfactants

Surfactants are surface active agents, and are defined as organic compounds that can enhance cleaning efficiency, emulsifying, wetting, dispersing, solvency, foaming/defoaming, and lubricity of water-based compositions. All surfactants share a common chemical structure: a hydrophilic (water-loving) “head” and a hydrophobic (oil-loving) “tail,” which is always a long (linear) chain of carbon atoms. Surfactants are generally classified into two groups: petroleum chemistry-derived or synthetic surfactants, and living-cell produced biosurfactants.

The primary function of surfactants is to enhance the performance properties of water-based formulations including a range of ingredients such as other surfactants, solvents, thickeners, alkalis/salts, chelating agents, foamers/defoamers and fragrances. They are grouped by their ionic charge into four groups: anionic, nonionic, cationic, and amphoteric surfactants. Anionic surfactants are the largest group accounting for approximately 34% of world production, and they exhibit superior wetting and emulsifying properties and tend to be higher-foaming materials.

Biosurfactants

Biosurfactants are surface-active substances synthesized by living cells such as bacteria, fungi and yeast. Biosurfactants are generally non-toxic, environmentally benign, and biodegradable. By comparison, chemical surfactants, which are usually derived from petroleum, are commonly toxic to health and ecosystems, and resist complete degradation. Biosurfactants have the properties of reducing the surface tension of a liquid, reducing the interfacial tension between two liquids, emulsifying/solubulizing water-insoluble or poorly soluble substances, stabilizing oil-in-water emulsions, demulsification and promoting foaming.

Rhamnolipids

Rhamnolipids are members of the glycolipid biosurfactants and were first identified from Pseudomonas sp. in 1949. Since then, numerous microorganisms including bacteria, fungi and yeast have been reported to produce rhamnolipids. Pseudomonas aeruginosa species of soil bacteria have been identified as the most frequently isolated rhamnolipid producers.

An amphiphilic rhamnolipid molecule is composed of two moities. One part is a hydrophilic sugar, mono- or di-rhamnose, and the other part is a hydrophobic lipid possessing one or two 3-hydroxy fatty acid residues. These residues may either be both fully saturated or one may be saturated and the other unsaturated, with either one or two double bonds. The lipid moiety is attached to the sugar by O-glycosidic linkage while the two 3-hydroxy acyl groups are joined together by the formation of an ester bond.

The structure diversity of rhamnolipids is determined by the number of rhamnose (one or two) and fatty acids (one or two), and the fatty acid composition. The length of the constituent fatty acids and their combinations has been found to be largely variable. To date, over 40 different rhamnolipid congeners have been described, though Rha-C₁₀-C₁₀ and Rha-Rha-C₁₀-C₁₀ are typically found to be the dominant components in a naturally occurring mixture. Rhamnolipids are commonly classified into two groups: monorhamnolipids and dirhamnolipids.

The functions of monorhamnolipids and dirhamnolipids include that of a natural surfactant, emulsifier, foaming/wetting agent, solubilizer, bactericide and/or fungicide, and/or anionic complexation agent.

Biosurfactants may be used as bioavailability enhancers in some pharmaceutical applications. For example, newly discovered molecules with potential as active pharmaceutical ingredients (APIs) often exhibit low solubility and/or low permeability, which limits their use as marketable drug ingredients. In fact, 40-70% of new chemical entities are classified as either biopharmaceutics classification system (BCS) Class II, having low solubility but high permeability (e.g., human absorption), or as BCS Class IV, having both low solubility and low permeability. To address these limitations, drug formulations and delivery systems may use lipophilic surfactants as solution components, for instance to provide the necessary solubilization, emulsification, and permeability for new, potential APIs that fall into BCS Classes II and IV.

One strategy for increasing bioavailability involves lipid-based drug delivery systems, which leverage a chosen surfactant or co-surfactant to solubilize the active ingredient into either an emulsion or micelles or liposomes, to maintain stability in the new form, and then to help it absorb (permeabilize) into the human drug recipient. Matching the drug with a surfactant is important; lipophilic surfactants need to be matched with lipophilic active ingredients. Ultimately the complete drug delivery system is a set of tested, compatible ingredients which include the drug, surfactant(s) and other excipients, such as solvents.

Another strategy to increase bioavailability uses self-emulsifying drug delivery systems (SEDDS), which include surfactants, oils and sometimes co-solvents, along with a solubilized and lipid-based “preconcentrate” of the drug (all contained within a soft-gel capsule). These systems are designed to automatically form emulsions when they reach the gastrointestinal tract, delivering the drug in solubilized, small droplet form for maximum absorption inside the intestine.

Biosurfactants also may be used as drug delivery system components. Biosurfactants, such as rhamnolipids and surfactin, are not petroleum-based, but are bio-synthesized from natural organisms, and thus have the advantage of sustainability, biodegradability and low-toxicity. Rhamnolipid biosurfactants are classified as lipophilic, and have especially strong interfacial tension characteristics.

Structures and Antibiotic Activity of Ramoplanin

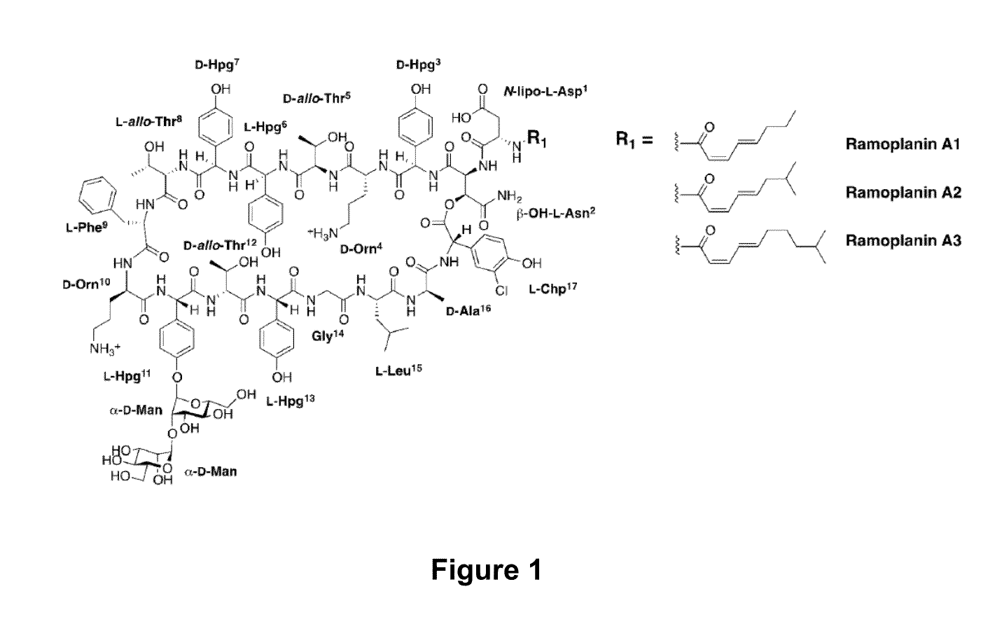

Ramoplanins are produced by Actinoplanes strain ATCC33076 and naturally occur as a mixture of at least three forms, A1, A2 and A3, with A2 being the most abundant. FIG. 1 illustrates the chemical structure of ramoplanin, in accordance with various embodiments. The 17 amino acid non-ribosomally synthesized lipodepsipeptide ramoplanins vary only in the length of the attached lipid chain. Structural characteristics of ramoplanins include the presence of numerous nonproteinogenic amino acid residues such as beta hydroxy-asparagine (β-OH-LAsn), 4-hydroxyphenylglycine (Hpg), D- and L-allo-threonines, D-ornithine and 3-chloro-4-hydroxyphenylglycine (L-Chp). Seven of the 17 amino acids have the D configuration and six of the residues are Hpg or the chlorinated derivative Chp. Ramoplanin has an α-1,2-dimannosyl moiety appended to Hpg¹¹.

The disaccharide group of ramoplanin does not contribute to antimicrobial activity. In fact, enduracidin, which is structurally and functionally closely related to ramoplanin, contains no sugar attachment and still has comparable activity to that of ramoplanin. Therefore, the disaccharide appendage likely primarily contributes to improved aqueous solubility and helps define the amphipathic nature of these peptides.

Ramoplanins A1, A2, and A3 possess essentially identical antibiotic activities. For simplicity, the peptides are referred to herein in the singular. Ramoplanin exhibits potent in vitro and in vivo antibacterial activity against a wide spectrum of major Gram-positive pathogenic infections, including clinically important VRE, MRSA, and vancomycin intermediate-resistant Clostridium difficile. Ramoplanin was reported to be 4 to 8-fold more active than vancomycin versus 500 strains Gram-positive species. For comparison, typical MICs for vancomycin toward sensitive strains of S. aureus range from 0.5 to 2 μg/ml. Since its discovery over two decades ago, no clinical resistance to ramoplanin has been reported. Ramoplanin has progressed to late stage clinical trials for the treatment of C. difficile-associated disease in the gastrointestinal (GI) tract.

Ramoplanin is a promising candidate to replace vancomycin, the last resort antibiotic for CDI. However, ramoplanin is poorly tolerated after intravenous or intramuscular administration, and is unstable in the bloodstream and not absorbed systemically when it is orally administered. Also, ramoplanin cannot be administered parenterally, owing to hemolysis and injection site swelling. Thus, the in vitro synergistic activity of ramoplanin in combination with rhamnolipids described herein may improve not only the efficacy of ramoplanin treatment, but also may avoid the current problems associated with administrating the antibiotic.

Antibacterial Mechanism of Action of Ramoplanin

Ramoplanin acts as an inhibitor of peptidoglycan (PG) biosynthesis, disrupting glycan chain polymerization by binding and sequestering Lipid II, a PG precursor indispensable for production of bacterial cell wall. Recently, a U-shape-like X-ray crystal structure of a dimer of ramoplanin A2 was reported. Based on the crystal structure, a model was proposed for how ramoplanin may recognize the Lipid II target in the context of the bacterial membrane. In brief, and without being bound by theory, the diphosphate group of Lipid II may be set at the membrane surface, where a salt bridge may be formed between the diphosphate moiety and Orn¹⁰ of ramoplanin. Without being bound by theory, the present disclosure may indicate that the most important role of rhamnolipids is as a biosurfactant excipient in synergistically enhancing the antibiotic activity of ramoplanin against superbugs VRE and MDR-CD.

Antibacterial Activity of Rhamnolipids

Rhamnolipids have been shown active against many microorganisms including Gram-negative and Gram-positive bacteria, phytopathogenic fungi, algae, viruses as well as amoeba. A monorhamnolipid predominant mixture usually prepared to contain over 50 to 90% of Rha-C₁₀-C₁₀ is more active than a dirhamnolipid predominant mixture usually prepared to contain over 50 to 90% of Rha-Rha-C₁₀-C₁₀. The minimum inhibitory concentrations of a mixture of rhamnolipid congeners were determined for some bacterial strains such as Methicillin-resistant Staphylococcus aureus ATCC33591 (MICs=50 μg/ml), Staphylococcus aureus ATCC6538 (MICs=32 and 128 μg/ml), Staphylococcus epidermidis ATCC11228 (MICs=32 and 8 μg/ml), and Clostridium perfringens ATCC486 (MICs=128 and 256 μg/ml). Without being bound by theory, the discrepancy in MICs for the same bacterial strains may be due to the variation of the purity and composition of rhamnolipid samples used and the quantification methods for rhamnolipid samples.

Antimicrobial Mechanisms of Action of Rhamnolipids

The antimicrobial mechanisms of action of rhamnolipids are poorly understood. The main mode of action of rhamnolipids against zoospore-producing phytopathogenic fungi has been better characterized to directly lyze zoospores via the interaction of rhamnolipids within plasma membranes of the zoospore. Bacterial cytoplasmic membranes are the primary site of surfactant action. Rhamnolipids are responsible for the membrane surface protein leakage due to alteration of the membrane permeability by possible aggregation and formation of transmembrane pores channeling to the periplasm. Rhamnolipids were able to cause the changes of cell surface hydrophobicity and loss of lipopolysaccharide (LPS) from the outer membrane. Bacteria with smooth-form LPS are resistant to the action of hydrophobic antibiotics. In contrast, bacteria without LPS are more susceptible to the action of hydrophobic antibiotics.

Rhamnolipids increase the negatively charged phospholipid cardiolipin in the presence of specific thiosulfonic alkyl esters. The changes in phospholipid's composition of bacterial membrane may lead to increased susceptibility of bacteria to some antibiotics. Mass spectrometric study of rhamnolipid biosurfactant indicates that the formation of stable supramolecule complexes of rhamnolipids with membrane phospholipids is a possible molecular mechanism of their antimicrobial activity.

### EXAMPLES

### Example 1

### Production and Purification of Rhamnolipids

In various embodiments, any source of rhamnolipids may be used to practice this disclosure no matter the origin of their producing microorganisms, preparation methods and composition and ratio of the rhamnolipid congeners in a mixture.

Unless otherwise stated, production and purification of rhamnolipid samples from the fermentation broths were according to publicly available standard protocols and optimized for column purification, TLC preparation and HPLC purification, LC-MS and MS/MS analyses to achieve and obtain the desired purities of rhamnolipids.

Rhamnolipids naturally occur as a mixture of dirhamnolipid and monorhamnolipid congeners regardless of microbial producers and fermentation media. Typically, dirhamnolipids and monorhamnolipids with a fatty acid chain length of 10 carbons (Rha-Rha-C₁₀-C₁₀ and Rha-C₁₀-C₁₀) are predominant in the naturally occurring mixture. After preparation by repeated acidic precipitation and solvent extraction, followed by multiple steps of column chromatography purification, different purity levels of rhamnolipids may be obtained. Isolation of a single congener from the mixture of rhamnolipids is technically and practically achievable but it is not cost effective. For this reason, practices in antimicrobial activity tests have been commonly conducted with a mixture of rhamnolipid congeners that were purified from fermentation broth and composed of Rha-Rha-C₁₀-C₁₀ or Rha-C₁₀-C₁₀ or both as predominant congeners as well as minor or trace amounts of other rhamnolipid congeners.

In various embodiments, LC-MS and MS/MS analysis may be used to readily detect the distinct rhamnolipid congeners in a fractionated and relatively pure sample preparation. The purity level of rhamnolipid preparation is important for accurate quantification, dosage and maximal elimination of any potential interference caused by the contamination of impure substances such as spent medium and an array of uncharacterized bacterial metabolites. For pharmaceutical, cosmetic and food additive uses, the high purity and quality of rhamnolipids (purity level from 90% to 99%) are required to meet regulatory standards.

### Example 2

### Determination of Purity, Concentration and Composition of Rhamnolipids

The purity and concentration of all rhamnolipid samples used in bioassays as disclosed herein were determined by TLC or TLC and HPLC using the internal highly pure (95%) rhamnolipid preparations as standards (now commercially available from Sigma-Aldrich). The compositions of all rhamnolipid samples used in bioassays were determined by LC-MS or LC-MS plus MS/MS analyses.

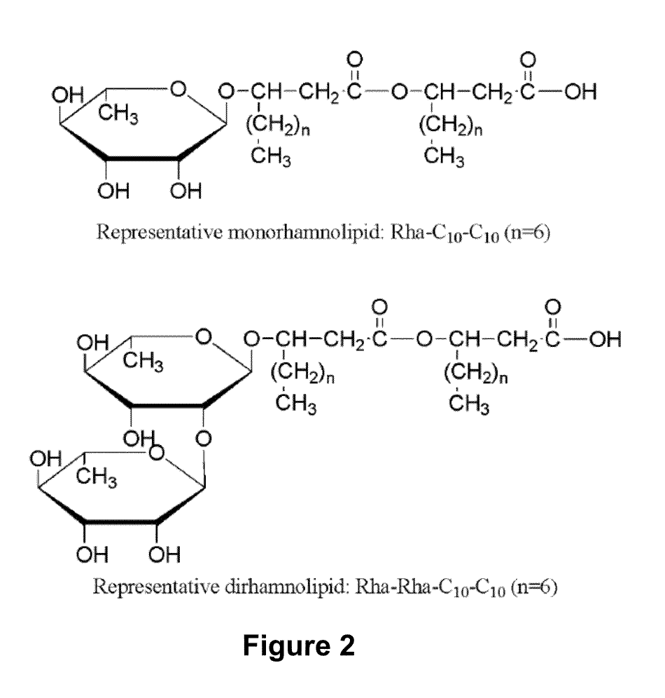

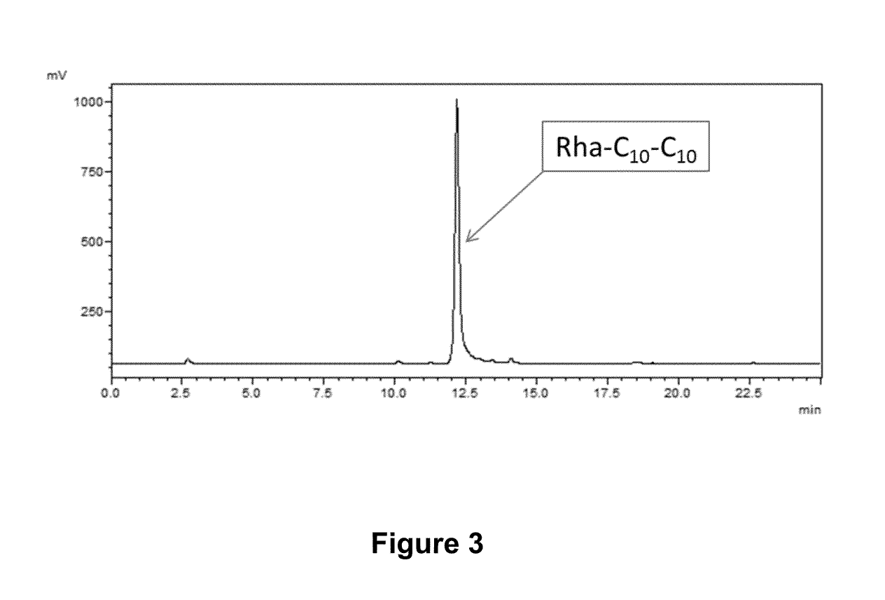

FIG. 2 illustrates the chemical structure of representative rhamnolipids, and FIG. 3 illustrates the HPLC profile of 95% pure rhamnolipids R95M, in accordance with various embodiments. R95M is defined herein to be a rhamnolipid sample that is at least 95% pure, and the monorhamnolipid congener, Rha-C₁₀-C₁₀, is predominant and at least 90% in total. The ratio of monorhamnolipids to dirhamnolipids is approximately 5:1. The minor or trace amounts of all other rhamnolipid congeners are less than 5%.

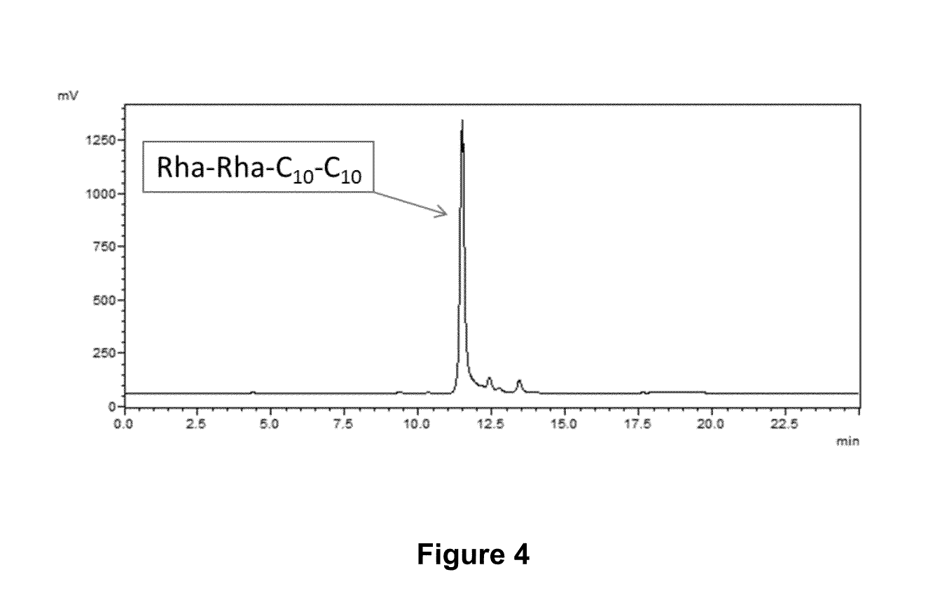

FIG. 4 illustrates the HPLC profile of 95% pure rhamnolipids R95D, in accordance with various embodiments. As used herein, R95D is defined to be a rhamnolipid sample that is at least 95% pure, and the dirhamnolipid congener, Rha-Rha-C₁₀-C₁₀ is predominant and at least 90% in total. The ratio of dirhamnolipids to monorhamnolipids is approximately 5:1. The minor or trace amounts of all other rhamnolipid congeners are less than 5%.

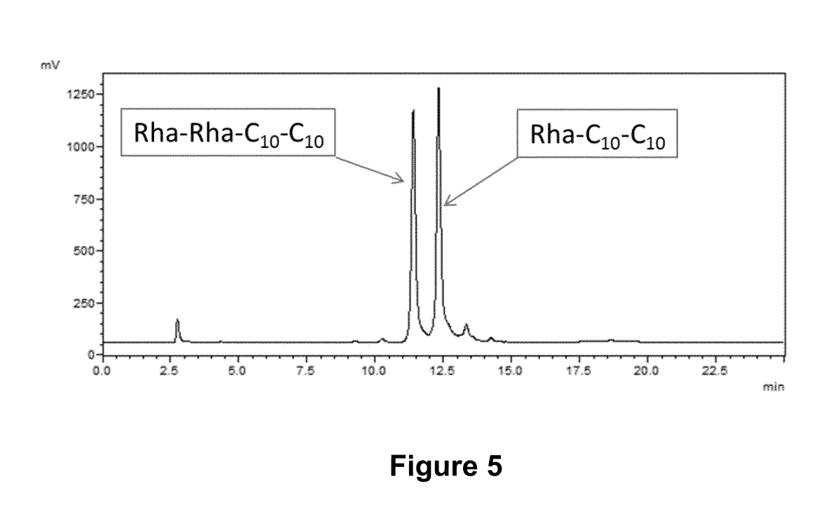

FIG. 5 illustrates the HPLC profile of 95% pure rhamnolipids R95DM, in accordance with various embodiments. As used herein, R95DM is defined to be a rhamnolipid sample that is at least 95% pure, and that contains approximately 50% each of monorhamnolipids and dirhramnolipids. The dirhamnolipid congener, Rha-Rha-C₁₀-C₁₀, is at least 40% in total. The monorhamnolipid congener, Rha-C₁₀-C₁₀, is at least 40% in total. The rest are the other rhamnolipid congeners.

### Example 3

### MIC Determination for MRSA/VISA, VRE and MDR-CD

Unless otherwise stated, the antibiotics used in the various examples described herein, including daptomycin, vancomycin, teicoplanin, tylosin and ramoplanin, were purchased from Sigma-Aldrich. All chemicals for general media and pre-prepared media as well as solvents, were purchased from Sigma, VWR and Fisher.

Gram-positive pathogenic bacteria strains Staphylococcus aureus ATCC29213, Staphylococcus aureus ATCC700787, Enterococcus faecium ATCC700221, and Clostridium difficile ATCC BAA1382 were obtained from the American Tissue Culture Collection (ATCC) and used as received. The characteristics for these four strains are listed in Table 1.

TABLE 1
Characteristics of the Gram-positive pathogenic strains used in MIC
and FICI determinations

| Strains | MRSA/VISA | VRE | MDR | References |
|---|---|---|---|---|
| Staph. aureus ATCC29213 | | | | ATCC |
| (semi-resistant) | | | | |
| Staph. aureus ATCC700787 | yes | | | ATCC |
| Enterococcus faecium | | yes | | ATCC |
| ATCC700221 | | | | |
| Clostridium difficile | | | yes | ATCC |
| ATCCBAA1382 | | | | |

All chemicals and other media were used as received, unless otherwise noted. Aerobic bacteria liquid growth media (Cation Adjusted Mueller Hinton II Broth) was obtained from Teknova, Inc. (Hollister, Calif.). Mueller Hinton II Agar and BBL Brucella Agar were obtained from BD Diagnostics (Franklin Lakes, N.J.) and prepared according to the manufacturers' directions. Anaerobic brucella broth was obtained from Anaerobe Systems (Morgan Hill, Calif.). Hemin was obtained from Alfa Aesar Inc. (Ward Hill, Mass.). Vitamin K₁ was obtained from MP Biomedicals, Inc. (Santa Ana, Calif.). Ampicillin was obtained from Sigma Aldrich (St. Louis, Mo.). Defibrillated sheep blood was obtained from Lampire Biological Laboratories (Pipersville, Pa.). Sterile clear polystyrene 96 well microplates (Corning 3628) were obtained from Corning Inc. (Tewksbury, Mass.). Six well clear polystyrene plates and GasPak™ EZ Anaerobe System anaerobic growth pouches were obtained from BD Diagnostics (Franklin Lakes, N.J.). Optical Density at 600 nM (OD₆₀₀) values were measured using a Biomate 3 spectrophotometer (Thermo Fisher Scientific) for cuvette samples or using a BioTek Synergy 4HT microplate reader (BioTek Inc., Winooski, Vt.) for microplate samples. Handling of concentrated anaerobic bacterial strains was performed in a glove box (PlasLabs, Lansing, Mich.) under an anaerobic atmosphere (10% H₂, 10% CO₂, 80% N₂, produced by AirGas, Vancouver, Wash.). Anaerobic conditions were monitored using BD resazurin indicator strips from BD Diagnostics (Franklin Lakes, N.J.).

### Example 4

### MIC Determinations for Aerobic Bacterial Strains (S. Aureus and E. Faecium)

Minimum growth inhibitory concentration (MIC) levels for each test compound were quantitated using a Meuller-Hinton broth (MHB) microdilution protocol as outlined in the Clinical and Laboratory Standards Institute (CLSI) publication M07-A9 section 10.4. In brief, 10 μl of thawed bacteria cryo-stocks were seeded into 7 ml of sterile MHB and grown at 37° C. with shaking to an OD₆₀₀ of 0.6AU (as measured in a 1 cm cuvette). A 1 μl aliquot was then streaked onto a 10 cm Meuller-Hinton agar plate and incubated at 37° C. overnight.

The following morning, drug test plates were prepared by adding 88 μl of sterile MHB to each well of a clear sterile polystyrene 96 well microplate. To this 11 μl of freshly prepared 10× drug solution were added to appropriate test wells of each plate.

Following preparation of drugged microplates, isolated isomorphic bacterial colonies were picked from the agar plate and resuspended in 1 ml sterile MHB. The OD₆₀₀ of the resulting suspension was measured in a cuvette and diluted to 0.132AU (equivalent to 0.5 McFarland) to create the final bacterial inoculum solution. Eleven microliters of this solution were then added to appropriate wells of the microplate. On each microplate 8 inoculated wells were left without drug and 8 wells were left without both drug and inoculum solutions to serve as negative and positive control populations respectively. The microplates were then sealed with adhesive seals and incubated for 24 hours at 35° C. with no shaking.

After 24 hours, the seals were removed and growth quantitated by both OD₆₀₀ values and visual assessment of growth levels. OD₆₀₀ values were converted to percent growth by normalizing to average positive (no inoculum, no drug) and negative (no drug, with inoculum) well values for each plate. After assessment of growth, plates were re-sealed and returned to the 35° C. incubator. Growth was assessed at 48 and 72 hour time points using an identical procedure.

Each test compound was tested in triplicate at each concentration reported. Performance of the assay was determined by using ampicillin as a reference compound at the following final concentrations: 120, 60, 30, 15, 7.5, 3.75, 1.88, 0.94, 0.47 and 0.23 μg/ml.

### Example 5

### MIC Determinations for Anaerobic Bacterial Strains C. Difficile

MIC values for each test compound were determined using a supplemented Brucella Agar dilution protocol based on that described in CLSI publication M11-A8 12.2. In brief, 500 μl of C. difficile cryo-stock was thawed under anaerobic conditions and added to 5 ml of anaerobic brucella broth in a test tube with a gas tight lid. This starter culture was then grown overnight at 37° C. with shaking to an OD₆₀₀ of approximately 0.6. A 1 μl aliquot of this culture was then streaked onto a 10 cm Supplemented Brucella Agar (Brucella agar+5 μg/ml hemin, 1 μg/ml vitamin K1 and 5% v/v laked sheep blood) plate and incubated at 37° C. overnight in a clear sealed anaerobic growth pouch equipped with an oxygen indicator.

The following morning, fresh agar dilution drug plates were prepared as follows: a solution of brucella agar with 5 μg/ml hemin and 1 μg/ml vitamin K1 was autoclaved and chilled to 50° C. followed by addition of 5% (v/v) laked sheep blood (defibrillated sheep blood which has been subjected to a freeze-thaw cycle). This mixture was then used to dilute 10× test samples (400 μl 10× sample added to 3.6 ml molten supplemented brucella agar) to a 1× final concentration. The solution was mixed by pipetting and 2 ml was transferred to a single well of a 6-well tissue culture plate. Each test plate also included an un-drugged well to serve as a full growth control. After agar was added to all wells, plates were allowed to solidify in a biosafety cabinet for at least 30 minutes.

Following preparation of drugged 6-well agar plates, isolated isomorphic bacterial colonies were picked from the 10 cm agar plate and resuspended in 1 ml sterile anaerobic brucella broth. The OD₆₀₀ of the resulting suspension was measured in a cuvette and diluted with additional sterile broth to 0.132AU (equivalent to 0.5 McFarland) to create the final bacterial inoculum solution. Each test well of the 6 well plate was inoculated at four distinct marked locations with 2 μl of the inoculum solution. The plates were then placed into clear sealed anaerobic growth pouches equipped with oxygen indicators and incubated at 37° C. Growth at each inoculation location was scored visually at 24 hours, 48 hours and 72 hours. The MIC for each compound was reported as the lowest tested concentration of each compound resulting in decreased growth levels as compared with an untreated test well. The performance of this assay was also assessed by using ampicillin as a reference compound at the following concentrations: 64, 32, 16, 8, 4, 2, 1, 0.5, 0.25 and 0.125 μg/ml.

### Example 6

### MICs' Determination for Agae Proprietary Rhamnolipids R95M, R95D and R95DM Alone or Individual Combination with Ramoplanin Against MRSA/VISA, VRE, MDR-CD and a Semi-Resistant Staphylococcus Aureus ATCC29213

The concentrations of the drugs and rhamnolipids are listed in Tables 2 and 3, below.

TABLE 2
Determination of the concentration of R95M for use in the synergistic
combination assay with ramoplanin, vancomycin, and daptomycin
Final concentrations of ramoplanin + R95M (μg/ml)

| | | |
|---|---|---|
| 0.2 + 0.08 (1)* | 0.25 + 0.1 (2) | 0.3 + 0.12 (3) |
| 0.2 + 0.4 (4) | 0.25 + 0.5 (5) | 0.3 + 0.6 (6) |
| 0.2 + 0.8 (7) | 0.25 + 1 (8) | 0.3 + 1.2 (9) |
| 0.2 + 4 (10) | 0.25 + 5 (11) | 0.3 + 6 (12) |
| 0.2 + 8 (13) | 0.25 + 10 (14) | 0.3 + 12 (15) |
| 0.2 + 16 (16) | 0.25 + 20 (17) | 0.3 + 24 (18) |
| 0.2 + 32 (19) | 0.25 + 40 (20) | 0.3 + 48 (21) |

Notes:
*indicated the culture number. Starting inhibition was observed in culture number 8 (in red pen) and thereafter. Complete inhibition was observed in culture number 17 (in bold red pen). Strain Staph. aureus ATCC29213 was used in bioassay.

TABLE 3
Final concentrations of rhamnolipids, ramoplanin, vancomycin and
daptomycin used in MIC determinations

| Compounds | Concentrations (μg/ml) | Strains |
|---|---|---|
| ramoplanin | 0.125, 0.25, 1, 2, 4, 8, 16, 32 | Staph. aureus |
| ramoplanin + R95M | 0.125 + 20, 0.25 + 20, 1 + 20, | ATCC29213 |
| | 2 + 20, 4 + 20, 8 + 20, | Staph. aureus |
| | 16 + 20, 32 + 20 | ATCC700787 |
| | | E. faecium |
| | | ATCC700221 |
| ramoplanin + R95M | 0.0125 + 20, 0.015625 + 20, | C. difficile |
| | 0.03125 + 20, 0.0625 + 20, | ATCCBAA1382 |
| | 0.25 + 20 | |
| vancomycin | 0.125, 0.25, 1, 2, 4, 8, 16, 32 | Staph. aureus |
| vancomycin + R95M | 0.125 + 20, 0.25 + 20, 1 + 20, | ATCC29213 |
| | 2 + 20, 4 + 20, 8 + 20, | Staph. aureus |
| | 16 + 20, 32 + 20 | ATCC700787 |
| daptomycin | 0.125, 0.25, 1, 2, 4, 8, 16, 32 | E. faecium |
| daptomycin + R95M | 0.125 + 20, 0.25 + 20, 1 + 20, | ATCC700221 |
| | 2 + 20, 4 + 20, 8 + 20, | |
| | 16 + 20, 32 + 20 | |
| R95M | 1.95, 3.9, 7.8, 15.6, 31.25, 62.5, | Staph. aureus |
| | 125, 250, 500 | ATCC29213 |
| R95D | 1.95, 3.9, 7.8, 15.6, 31.25, 62.5, | Staph. aureus |
| | 125, 250, 500 | ATC700787 |
| R95DM | 1.95, 3.9, 7.8, 15.6, 31.25, 62.5, | E. faecium |
| | 125, 250, 500 | ATCC700221 |
| | | C. difficile |
| | | ATCCBAA1382 |
| R95M | 62.5, 125, 250, 500, 1000 | C. difficile |
| | | ATCCBAA1382 |

Using the MIC determination protocols described in detail in this disclosure, the MICs for individual rhamnolipid preparation alone or its combination with ramoplanin against the representative superbugs tested were shown in Tables 4, 5, 6, and 7.

TABLE 4
MICs (μg/ml) determined for single drug or individual drug combined with
rhamnolipids R95M against strains Staph. aureus, MRSA/VISA and VRE

| | | | Strains | | |
|---|---|---|---|---|---|
| | | | Staph. | Staph. | Enterococcus |
| Compounds | | Exposure | aureus | aureus | faecium |
| Single | Combination | (hours) | ATCC29213 | ATCC700787 | ATCC700221 |
| ramoplanin | | 24 | 2 | 2 | 2 |
| | | 48 | 2 | 2 | 2 |
| R95M | | 24 | 500 | 250 | 125 |
| | | 48 | 500 | 250 | 250 |
| | ramoplanin + R95M | 24 | 0.25 | 2 | 0.25 |
| | | 48 | 0.25 | 2 | 0.25 |
| R95D | | 24 | 500 | 500 | 125 |
| | | 48 | >500 | 500 | >500 |
| R95DM | | 24 | 500 | 500 | 125 |
| | | 48 | 500 | 500 | 125 |
| vancomycin | | 24 | 2 | 8 | >32 |
| | | 48 | 2 | 8 | >32 |
| | vancomycin + R95M | 24 | 2 | 4 | >32 |
| | | 48 | 2 | 4 | >32 |
| daptomycin | | 24 | 2 | 4 | 8 |
| | | 48 | 2 | 8 | 8 |
| | daptomycin + R95M | 24 | 2 | 8 | 16 |
| | | 48 | 2 | 8 | 16 |

TABLE 5
MICs, FICs and FICIs (μg/ml) determined for single drug or individual
drug combined with rhamnolipids R95M against strains MRSA/VISA and VRE

| | | | Staph. aureus | | | Enterococcus faecium | | |
|---|---|---|---|---|---|---|---|---|
| | | | ATCC700787 | | | ATCC700221 | | |
| Single | Combination | | MIC | FICI | Effects | MIC | FICI | Effects |
| ramoplanin | | 24 | 2 | 1 | | 2 | 0.125 | |
| | | 48 | 2 | 1 | | 2 | 0.125 | |
| R95M | | 24 | 250 | 0.08 | | 125 | 0.16 | |
| | | 48 | 250 | 0.08 | | 250 | 0.08 | |
| | ramoplanin + | 24 | 2 + 20 | 1.08 | antagonistic | 0.25 + 20 | 0.285 | synergistic |
| | R95M | 48 | 2 + 20 | 1.08 | antagonistic | 0.25 + 20 | 0.208 | synergistic |
| vancomycin | | 24 | 8 | 0.5 | | >32 | >1 | |
| | | 48 | 8 | 0.5 | | >32 | >1 | |
| | vancomycin + | 24 | 4 + 20 | 0.58 | additive | >32 + 20 | >1.16 | antagonistic |
| | R95M | 48 | 4 + 20 | 0.58 | additive | >32 + 20 | >1.08 | antagonistic |
| daptomycin | | 24 | 4 | 2 | | 8 | 2 | |
| | | 48 | 8 | 1 | | 8 | 2 | |
| | daptomycin + | 24 | 8 + 20 | 2.08 | antagonistic | 16 + 20 | 2.16 | antagonistic |
| | R95M | 48 | 8 + 20 | 1.08 | antagonistic | 16 + 20 | 2.08 | antagonistic |

TABLE 6
MIC (μg/ml) determined for ramoplanin or rhamnolipids R95M alone,
and ramoplanin in combination with rhamnolipids R95M against
multidrug-resistant C. difficile (MDR-CD)

| Compounds | | | |
|---|---|---|---|
| Single | Combination | Exposure (hours) | MIC |
| ramoplanin | | 24 | 0.25 |
| | | 48 | 0.25 |
| R95M | | 24 | 62.5 |
| | | 48 | 250 |
| | ramoplanin + R95M | 24 | 0.0125 |
| | | 48 | 0.0125 |
| R95D | | 24 | 250 |
| | | 48 | 500 |
| R95DM | | 24 | 250 |
| | | 48 | 500 |

TABLE 7
MICs, FICs and FICIs (μg/ml) determined for ramoplanin or rhamnolipids R95M alone, and ramoplanin
in combination with rhamnolipids R95M against multidrug-resistant C. difficile (MDR-CD)

| | | | | | | | FICI | Effect |
|---|---|---|---|---|---|---|---|---|
| Compounds | | Exp. | MIC | MIC | FIC | FIC | (ramoplanin & | (ramoplanin + |
| Single | Comb. | (hours) | (individ.) | (comb.) | (ramoplanin) | (R95M) | R95M) | R95M) |
| ramoplanin | | 24 | 0.25 | 0.0125 | 0.05 | | | |
| | | 48 | 0.25 | 0.0125 | 0.05 | | | |
| R95M | | 24 | 62.5 | 20 | 0.32 | 0.32 | | |
| | | 48 | 250 | 20 | 0.08 | 0.08 | | |
| | ramoplanin + | 24 | | 20 + 0.0125 | | | 0.37 | Synergistic |
| | R95M | 48 | | 20 + 0.0125 | | | 0.13 | Synergistic |

The MICs (Tables 4 and 6), FICs, and FICIs (Tables 5 and 7) results provide the strongest support and evidence that there are beneficial synergistic activity between rhamnolipids R95M (See, e.g. FIG. 3) and ramoplanin against vancomycin resistant Enterococcus faecium ATCC700787 and multidrug-resistant Clostridium difficile ATCC-BAA1382. Also, MICs results for rhamnolipids R95M or R95D (FIG. 4) or R95DM (FIG. 5) alone are favorable to their broad applications at the final concentration of 125 to 500 μg/ml (Tables 4 and 6) in the treatment of, decontamination of, and prevention of the superbug infections including but not limited to VRE and MDR-CD.

### Example 7

### Applications of Rhamnolipids

The current disclosure enables efficient and effective applications of rhamnolipids related to the treatment, decontamination, and prevention of superbug infections. These applications may include, but not be limited to, (1) the formulation of ramoplanin- and rhamnolipid-based clinic drugs, anti-superbug detergents and disinfectants; (2) the formulation of rhamnolipid-based green, anti-superbug detergents, disinfectants, bandages and wipes; (3) cost-effective utilization of green, rhamnolipid biosurfactant aqueous solutions at the right concentration and combination for a routine cleaning of health care facilities, materials and environments to prevent and get rid of the superbugs; (4) cost-effective utilization of green, rhamnolipid biosurfactant aqueous solutions at correct concentration and combination for periodical hygiene and sanitation of animal farms, factory farms and associated environments; (5) cost-effective utilization of green, aqueous rhamnolipid biosurfactant solutions at the correct concentration and combination for cleaning up, decontamination and treatment of the drinking water, waste water, swimming pools, water plants, wells, ponds, rivers and lakes to prevent and get rid of the superbugs; (6) the formulation of cost-effective rhamnolipids- or rhamnolipids and ramoplanin-based animal feed additives targeting superbug-free agricultural organic food production; (7) cost-effective utilization of green, aqueous rhamnolipid biosurfactant solutions at the correct concentration and combination for cleaning, decontamination, and treatment of kitchen and restaurant facilities and waste oil pipelines, containers and collectors to prevent and get rid of the superbugs.

Specific, non-limiting additional applications include, but are not limited to the following.

1. Cosmetics Industry.

The biologic activity of rhamnolipids supports their application in dermatology. Disclosed herein are rhamnolipid-specific roles in combatting superbugs such as VRE and MDR-CD. Incorporation of rhamnolipids at the right concentration and combination as described herein may produce novel anti-superbug cosmetic products such as beauty products, creams, moisturizers, body lotions, make-up, lipstick, eyeshadow, denture cleaners, antiperspirants, lubricated condoms, baby products, foot care products, antiseptics, shampoo, soap, hair care products (antidandruff, hair color), skin care products (acne pads), anti-acid preparations, bath and shower products, toilet soap bars, liquid hand soap, toothpaste, contact lens solutions, deodorants, nail care products, shaving preparations, depilatory products, liposome, emulsions and cosmetic additives.

2. Household Products

Incorporation of rhamnolipids at the right concentration and combination as disclosed herein may produce novel anti-superbug household products (e.g., wetting, foaming and dispersing agents for detergent and cleaning formulations), heavy duty detergents, light duty liquids, prewash products, dishwashing products, fine fabric detergents and hard surface cleaners.

3. Pharmaceutical Industry

The disclosed rhamnolipid formulations may show synergistic activities when used in combination with various therapeutics (e.g., antibiotics, anticancer agents, etc.).

4. Ointments

Use of the disclosed rhamnolipid formulations in therapeutic ointments may produce novel anti-superbug ointment products, and may be used for wound healing, for instance. Three mechanisms of action of dirhamnolipids may be involved. First, dirhamnolipids stimulate bone marrow for significant increase in neutrophils and monocytes. Secondly, dirhamnolipids stimulate the proliferation of kerratinocytes which are helpful for the wound reepithelialization. Lastly, dirhamnolipids diminish fibrosis.

### Example 8

### Inhibition of MRSA/VISA, VRE and MDR-CD by Rhamnolipids R95D, R95M and R95DM

To examine the antimicrobial activity and determine the MICs with individual rhamnolipid preparations R95D, R95M and R95DM, each rhamnolipid sample was serially diluted to obtain the final concentrations from 1.95 to 500 or 62.5 to 1000 μg/ml as shown in Table 3. Following the standard assay protocols described in this disclosure, the MICs were obtained and shown in Table 4 and Table 6.

R95M (see, e.g., FIG. 3 and Table 4) was shown to be more active than R95D (see, e.g., FIG. 4 and Table 4) and R95DM (see, e.g., FIG. 5 and Table 4) against strains MRSA/VISA and VRE (Table 4). No significant differences between R95D and R95D/M were observed in all tested strains with respect to their MICs.

Monorhamnolipids had better antimicrobial activity than dirhamnolipids. However, it is worth noting that there is a significant discrepancy for the MIC values previously reported in the literature from one laboratory to another even though the same ATCC strains were used. These differences are most likely due to the complexity of the rhamnolipid sample preparation and characterization. Any inaccurate determination of the composition, concentration and combination would contribute to the observed differences. Because monorhamnolipids have better surfactant activity and are more potent as antibacterial agent, R95M (FIG. 3) was used in the combination studies for all MIC determinations reported herein.

### Example 9

### Inhibition of MRSA/VISA, VRE and MDR-CD by Lipoglycopeptide Antibiotic Ramoplanin

To examine the antimicrobial activity and determine the MICs with the lipoglycodepsipeptide antibiotic ramoplanin alone and establishment of the reference MICs values for comparison, a commercial ramoplanin sample from Sigma-Aldrich was serially diluted to obtain the final concentrations from 0.125 to 32 μg/ml or 0.0125 to 0.25 μg/ml as shown in Table 3. Following the standard assay protocols described in this disclosure, the MICs were obtained and shown in Table 4 and Table 6.

MICs for ramoplanin alone were as low as 2 μg/ml against two staphylococcal strains and one enterococcal strain tested (see, e.g., Table 4). Ramoplanin was more potent against MDR-CD than against staphylococcal strains, which is reflected by an eight fold decrease in its MIC (0.25 μg/ml) (see, e.g., Tables 4 and 6). The significant differences in MICs between strain MRSA and VRE for ramoplanin combined with rhamnolipids R95M may merely reflect the optimized antibiotic activity conferred by the combination other than the strain-dependence. In other words, VRE is inherently more susceptible to the presence of biosurfactant rhamnolipids with ramoplanin than MRSA.

### Example 10

### Inhibition of MRSA/VISA, VRE and MDR-CD by Combination of Ramoplanin with Rhamnolipids R95M

To determine the concentration of R95M for use in the synergistic combination with ramoplanin, bioassays were conducted with a series of mixtures of R95M plus ramoplanin against Staph. aureus ATCC29213. (see, e.g., Table 2)

The concentration of R95M used in the mixture ranged from 0.08 to 48 μg/ml (Table 2), while the concentrations of ramoplanin applied in the mixture varied from 0.2 to 0.3 μg/ml. The starting inhibitory concentration of R95M on Staph. aureus ATCC29213 was found in combination No. 8. (1 μg/ml). The complete inhibition of the bacterial growth was observed in combinations of 17 to 21.

The concentration of R95M in combination No. 17 (20 μg/ml) was coincidentally the same as its critical micelle concentration (CMC). Thus, the empirical concentration of 20 μg/ml for R95M was used as a final concentration in all synergistic combination assays with ramoplanin disclosed herein.

Combination of ramoplanin with R95M has shown remarkable synergistic effects on E. faecium and Staph. aureus ATCC29213 (see, e.g., Table 4). An eight fold decrease of ramoplanin in MIC was observed against both strains (Tables 4 and 5). In contrast, no synergistic activity has been found for the combination of vancomycin or daptomycin with R95M (see, e.g., Table 4).

The most interesting findings came in the combination of ramoplanin with R95M against MDR-CD (see, e.g., Tables 6 and 7). This combination dramatically diminished the MIC for ramoplanin against MDR-CD (see, e.g., Table 6). A twenty-fold decrease in MIC for ramoplanin against this strain was demonstrated in vitro.

The MIC data shown in Table 4 strongly supports that E. faecium ATCC700221 is resistant to both vancomycin and daptomycin. This disclosure provides the strongest evidence that a specific beneficial interaction between ramoplanin and R95M occurred and resulted in exceptionally high synergistic activity.

This finding has both fundamental and clinically practical importance to help eventually defeat life-threatening superbugs, in particular when the first front-line drugs like vancomyin and daptomycin have become ineffective. In addition, the findings presented herein show that R95M is more active against the MRSA/VISA and VRE than semi-resistant Staph. aureus ATCC29213. Thus, the antimicrobial activity of rhamnolipids R95M may be independent of the bacterial resistance mutations and evolution. Naturally produced, common rhamnolipid molecules may be exploited in systemic screenings of novel synergistic activities with clinical drug collections.

The extraordinary synergistic activity of ramoplanin in combination with rhamnolipids indicates that other peptide antibiotic candidates may offer the similar synergistic effects against the superbugs as observed for ramoplanin. Mannopeptimycin, an anti-superbug lipoglycopeptide antibiotic, also has a di-mannose residue appended to its peptide core. Therefore, mannopeptimycin may have excellent synergistic activity in combination with rhamnolipids against superbugs if a beneficial interaction between ramopanin and rhamnolipids occurs through the di-mannose moiety. In addition, the rhamnolipid biosurfactant excipient may have similarly antibiotic activity enhanced effects on the possibly biologically synthesized dimannosylated enduracidin as observed for its counterpart ramoplanin.

Thus, the combination of excellent water-soluble peptide antibiotic ramoplanin with a customized rhamnolipid biosurfactant produced significant synergistic activity against MDR-CD and VRE. The combination of ramoplanin and rhamnolipid biosurfactant has several advantages as a pharmaceutical composition. As rhamnolipids are extremely tolerant to harsh pH, temperature and salinity, they can remain active in the gastrointestinal environment. At such low concentration (20 μg/ml) in the combination, rhamnolipids' role serves as a truly beneficial biosurfactant rather than an active antibiotic ingredient. The skin-friendly, burn- and wound-healing properties of rhamnolipids will add value to topical applications of the combination of ramoplanin with rhamnolipids. Additionally, the enhanced efficacy of the ramoplanin and rhamnolipid combination may significantly reduce the dosage of ramoplanin required for achieving effectiveness in the bloodstream and, accordingly, lower the potential toxicity of high-dosage ramoplanin treatment. Finally, the combination formulation with ramoplanin and rhamnolipids may also result in the increased stability and extended half-life of ramoplanin. Furthermore, there are large numbers of past and current clinically effective antibiotics worldwide whose efficacy may be improved in rhamnolipid formulations, for example, in anti-cancer, anti-HIV and other therapeutic libraries.

The antimicrobial activity of rhamnolipids is typically dose-dependent. R95M performed best in all synergistic assays with ramoplanin among tested antibiotics. It is evident that, in the past, overemphasis of the antimicrobial activity of rhamnolipids has misled drug formulators to 1) regard rhamnolipids as an active pharmaceutical ingredient (API), and, as a consequence, to 2) avoid or ignore the potential benefits of rhamnolipids solely serving as a biosurfactant excipient in drug formulations. It is worth noting that at very low concentrations, rhamnolipids actually serve as a biosurfactant excipient in drug formulations rather than as an antibiotic. This simple change of perspective regarding the chemical properties of rhamnolipids has encouraged the inventor to exploit these small molecules in novel formulations of currently administered clinical drugs.

The results shown in Tables 4, 5, 6, and 7 strongly support the premise that a specific and beneficial interaction has occurred between ramoplanin and rhamnolipids or their complex with superbugs' cell membranes. The extraordinary synergistic activity of ramoplanin in combination with rhamnolipids is highly selective rather than random, because similar synergistic activity was not observed for vancomycin, daptomycin, or other tested clinic drugs. In addition, all prolonged assays (up to 72 hour exposure) of the combination did not change the MIC values. Thus, without being bound by theory, a rhamnolipid biosurfactant excipient may serve as a molecular stabilizer to retain a favorable conformation of active ramoplanin on target sites, or, alternatively, it may promote the formation of an optimal ramoplanin conformation by mutual, local interactions that prime the affinity binding of ramoplanin to its target, Lipid II.

Ramoplanin has excellent water solubility, and that that property precludes the need for a solubilizing enhancer to elevate its bioavailability in solution. However, without being bound by theory, rhamnolipids alternatively may prevent or reduce ramoplanin molecular aggregation and/or polymerization by acting as a peptide aggregation spacer or disrupter. Therefore, the increased efficacy observed in vitro may result from an improvement in ramoplanin dimerization that directs a higher concentration of the bioactive form of the antibiotic to its target.

The toxicity of rhamnolipids alone has been well documented in animals in U.S. EPA publications. Their acute oral toxicity and acute dermal toxicity in rats are LD50>5000 mg/kg. The toxicity of ramoplanin alone has been studied in animals and humans. Specifically, it was tested in humans at 200, 400 or 800 mg/kg doses administered twice daily, and was reported to be safe and effective in temporarily suppressing asymptomatic gastrointestinal carriage of vancomycin-resistant Enterococci. The toxicity of the novel formulation of RamoR95M antibiotic would likely be similar.

### Example 11

### Coadministration of Ramoplanin with Rhamnolipids R95M and Pharmaceutical Compositions Containing Ramoplanin and Rhamnolipids R95M

In some embodiments, the disclosed combinations of ramoplanin and ramnolipids may be coadministered to a subject to prevent, treat, or ameliorate a superbug infection. In an embodiment, the compounds may be administered to a subject locally or systemically. In some embodiments, a combination of ramoplanin and one or more ramnolipids may be administered parenterally, for instance subcutaneously, intravenously, or intramuscularly, or they may be administered orally or by inhalation. In various embodiments, such combinations may be used alone or in combination with other anti-bacterial agents. In some embodiments, the ramoplanin and ramnolipids may be administered in varying concentrations depending upon the infection's susceptibility to the compound being administered, the extent of the disease, whether the infection is latent or active, whether the infection is drug-resistant, and the general health of the subject.

In various embodiments, a combination of ramoplanin and one or more ramnolipids may be incorporated into a pharmaceutical composition. Embodiments of the present disclosure encompass any racemic, optically-active, polymorphic, tautomeric, or stereoisomeric form or mixture thereof, of a compound of the disclosure, which possesses the useful properties described herein.

In cases where compounds are sufficiently basic or acidic to form stable nontoxic acid or base salts, use of the compounds as pharmaceutically acceptable salts may be appropriate. Examples of pharmaceutically acceptable salts within the scope of embodiments herein include organic acid addition salts formed with acids that form a physiologically acceptable anion and inorganic salts.

Pharmaceutical compositions in accordance with embodiments of the disclosure may be prepared by combining the disclosed compounds with a solid or liquid pharmaceutically acceptable carrier and, optionally, with pharmaceutically acceptable adjuvants and excipients employing standard and conventional techniques. Solid form compositions include powders, tablets, dispersible granules, capsules, cachets and suppositories. A solid carrier may be at least one substance that may also function as a diluent, flavoring agent, solubilizer, lubricant, suspending agent, binder, tablet disintegrating agent, and encapsulating agent. Inert solid carriers include magnesium carbonate, magnesium stearate, talc, sugar, lactose, pectin, dextrin, starch, gelatin, cellulosic materials, low melting wax, cocoa butter, and the like. Liquid form compositions include solutions, suspensions and emulsions. For example, there may be provided solutions of the compounds disclosed herein dissolved in water and water-propylene glycol systems, optionally containing suitable conventional coloring agents, flavoring agents, stabilizers, and/or thickening agents.

In some embodiments, a pharmaceutical composition may be provided employing conventional techniques in unit dosage form containing effective or appropriate amounts of one or more active component. In various embodiments, the quantity of active component (compound) in a pharmaceutical composition and unit dosage form thereof may be varied or adjusted widely depending upon the particular application, the potency of the particular compound and the desired concentration. In an exemplary embodiment, the quantity of active component may range from 0.5% to 90% by weight of the composition. In specific, non-limiting examples, the pharmaceutical composition may include from about 0.05 μg/ml to about 60 μg/ml rhamnolipids, from about 60 μg/ml to about 125 μg/ml rhamnolipids, or from about

125 μg/ml to about 1000 μg/ml rhamnolipids.

In various embodiments, in therapeutic uses for treating, ameliorating, preventing, or combating a superbug infection in a subject, the compounds or pharmaceutical compositions thereof may be administered orally, parenterally, and/or by inhalation at a dosage to obtain and maintain a concentration or blood-level of active component in the animal undergoing treatment that is therapeutically effective. In an embodiment, such a therapeutically effective amount/dosage of active component may be in the range of from about 0.05 to about 1000 mg/kg, for instance, from about 0.05 to about 60 mg/kg, from about 20 mg/kg to about 60 mg/kg, from about 60 mg/kg to about 125 mg/kg, or from about 60 mg/kg to about 1000 mg/kg of body weight per day or per dose.

It is to be understood that the dosages may vary depending upon the requirements of the patient, the severity of the infection, the particular superbug strain, whether the infection is latent or active, the drug resistance of the strain, the duration of the infection being treated, and the particular compound being used. Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose also may be divided into multiple doses for administration, for instance, two to four times per day. In some embodiments, timed-release formulations may be used, and in particular embodiments, a timed-release formulation of one compound may be used in conjunction with an immediate-release formulation of a second compound, for instance in order to deliver one compound before another compound.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A method of treating a superbug infection in a subject comprising:
administering to the subject a therapeutically effective dose of ramoplanin in combination with a therapeutically effective dose of one or more rhamnolipids, thereby treating the superbug infection.

2. The method of claim 1, wherein the superbug comprises vancomycin-resistant Enterococci, Clostridium difficile, or multidrug-resistant Clostridium difficiles or Clostridium difficile infections.

3. The method of claim 2, wherein the vancomycin-resistant Enterococcus is E. faecium ATCC700221.

4. The method of claim 1, wherein the multidrug-resistant Clostridium difficile is C. difficile ATCC-BAA1382.

5. The method of claim 1, wherein the one or more rhamnolipids comprise monorhamnolipid Rha-C₁₀-C₁₀.

6. The method of claim 1, wherein the one or more rhamnolipids comprise dirhamnolipid Rha-Rha-C₁₀-C₁₀.

7. The method of claim 1, wherein the one or more rhamnolipids comprise monorhamnolipid Rha-C₁₀-C₁₀ and dirhamnolipid Rha-Rha-C₁₀-C₁₀.

8. The method of claim 1, wherein the therapeutically effective dose of the one or more rhamnolipids is from about 0.05 mg/kg to about 60 mg/kg.

9. The method of claim 1, wherein the therapeutically effective dose of the one or more rhamnolipids is from about 60 mg/kg to about 1000 mg/kg.

10. The method of claim 9, wherein the therapeutically effective dose of the one or more rhamnolipids is from about 60 mg/kg to about 125 mg/kg.

11. A pharmaceutical composition comprising ramoplanin, one or more rhamnolipids, and a pharmaceutically acceptable carrier.

12. The pharmaceutical composition of claim 11, wherein the one or more rhamnolipids comprise monorhamnolipid Rha-C₁₀-C₁₀, dirhamnolipid Rha-Rha-C₁₀-C₁₀, or monorhamnolipid Rha-C₁₀-C₁₀ and dirhamnolipid Rha-Rha-C₁₀-C₁₀.

13. The pharmaceutical composition of claim 11, wherein the one or more rhamnolipids comprises from about 0.05 μg/ml to about 60 μg/ml rhamnolipids.

14. The pharmaceutical composition of claim 11, wherein the one or more rhamnolipids comprises from about 60 μg/ml to about 125 μg/ml rhamnolipids.

15. The pharmaceutical composition of claim 13, the one or more rhamnolipids comprises from about 125 μg/ml to about 1000 μg/ml rhamnolipids.

16. A formulation for preventing superbug infections, comprising a bacteriocidally effective concentration of one or more rhamnolipids.

17. The formulation of claim 16, further comprising a bacteriocidally effective concentration of ramoplanin.

18. The formulation of claim 17, wherein the superbug comprises vancomycin-resistant Enterococcus, Clostridium difficile, or multidrug-resistant Clostridium difficile.

19. The formulation of claim 18, wherein the vancomycin-resistant Enterococcus is E. faecium ATCC700221 and/or wherein the multidrug-resistant Clostridium difficile is C. difficile ATCC-BAA1382.

20. The formulation of claim 16, wherein the one or more rhamnolipids comprise monorhamnolipid Rha-C₁₀-C₁₀, dirhamnolipid Rha-Rha-C₁₀-C₁₀, or monorhamnolipid Rha-C₁₀-C₁₀ and dirhamnolipid Rha-Rha-C₁₀-C₁₀.

21. The formulation of claim 17, wherein the formulation is a component of a drug, detergent, disinfectant, hygiene product, sanitation product, wipe, or bandage.

22. The formulation of claim 16, wherein the bacteriocidally effective concentration of one or more rhamnolipids comprises from about 125 to about 1000 μg/ml.

23. The formulation of claim 22, formulated for use in a laundry application, cosmetics application, dishwashing application, landscaping application, swimming pool application, sports grounds application, hospital setting, medical facility, kitchen facility; or for use in a pipeline, container, or collector contaminated with food oil; or for use in the decontamination of water, drinking water, waste water, water in water treatment plants, well water, pond water, or river water.
